(19) United States
(12) Patent Application Publication
(10) Pub. No.: US 2024/0081682 A1
(43) Pub. Date: Mar. 14, 2024

(54) SYSTEM FOR DIAGNOSING A FOOT CONDITION BY SCANNING THE FOOT AND A METHOD FOR DIAGNOSING THE FOOT CONDITION USING THE SAME

(71) Applicant: HBT Co., Ltd., Seongnam-si (KR)

(72) Inventor: Kyung Won PARK, Seoul (KR)

(21) Appl. No.: 17/944,521

(22) Filed: Sep. 14, 2022

(51) Int. Cl. A61B 5/107; A61B 5/00; G16H 70/60

(52) U.S. Cl. CPC A61B 5/1077; A61B 5/004; A61B 5/0064; A61B 5/1079; A61B 5/702; A61B 5/7282; G16H 70/60; G16H 50/30

(57) ABSTRACT

A system for diagnosing a foot condition by scanning the foot includes a shape data module for detecting various shapes of foots and storing shape data; a condition data module for detecting the foot condition or a foot related condition and storing the condition data; a foot standard database for generating a standard data of the food based on the data stored in the shape data module and a condition data module and storing the generated standard data; a shape scanning data module for generating a 3D shape data related to the foot shape by scanning the foot in 3D way; a condition measuring data module for measuring a foot condition by scanning the foot and generating a foot condition data; and a foot diagnosing data module for generating a foot data based on the data generated by the shape scanning data module and the condition measuring data module.

### BACKGROUND OF THE INVENTION

### 1. Field of the Invention

The present invention relates to a system for diagnosing a foot condition by scanning the foot and a method for diagnosing the foot condition using the same, in particular the system and the method capable of diagnosing a foot disease or a foot related disease by scanning a foot shape and detecting a reaction.

### 2. Description of the Related Art

A foot for supporting the weight of a human body may influence on a posture and be related to various diseases of the human body. Therefore, the foot health should be maintained and the healthy condition of the foot The condition of the foot needs to be measured for checking the foot healthy condition, and various skill related to the measurement of the foot are known in this art. Korean patent registration number 10-2133771 discloses an apparatus for measuring a foot. And Korean patent publication number 10-2021-0147484 discloses a method for measuring a foot shape. The foot pressure distribution or the foot reaction condition as well as the measurement of the foot shape should be detected for diagnosing the foot health. And a method for maintaining the foot health based on such pressure distribution or the reaction may be developed. But the prior art does not disclose this method.

The present invention for solving the problem has a purpose as following.

### PURPOSE OF THE INVENTION

An object of the present invention is to provide with a system for diagnosing a foot condition by scanning the foot and a method for diagnosing the foot condition using the same capable of diagnosing a foot condition or a foot related health condition by measuring the foot pressure distribution or a stimulus reaction condition as well as the foot shape in a course of scanning the foot.

### SUMMARY OF THE INVENTION

In one embodiment of the present invention, a system for diagnosing a foot condition by scanning the foot comprises a shape data module for detecting various shapes of foots and storing shape data; a condition data module for detecting the foot condition or a foot related condition and storing the condition data; a foot standard database for generating a standard data of the food based on the data stored in the shape data module and a condition data module and storing the generated standard data; a shape scanning data module for generating a 3D shape data related to the foot shape by scanning the foot in 3D way; a condition measuring data module for measuring a foot condition by scanning the foot and generating a foot condition data; and a foot diagnosing data module for generating a foot data based on the data generated by the shape scanning data module and the condition measuring data module.

In other embodiment of the present invention, the system further comprises a foot estimating index generating module for generating a foot condition index based on a reaction to a response of a pressure according to a ground contact of the foot and a stimulus.

In another embodiment of the present invention, a method for diagnosing a foot by scanning the foot comprises generating a standard database related to the foot shape or condition; obtaining a shape data by scanning the foot; measuring a contacting pressure and a stimulus response; generating an estimating index related to divided parts of the foot based on the contacting pressure and the stimulus response; comparing the estimating index with a data from the standard database; and generating a diagnosing data related to a foot related disease.

### BRIEF DESCRIPTION OF THE DRAWINGS

FIG. 1 shows an exemplary embodiment of a system for diagnosing a foot condition by scanning the foot according to present invention.

FIG. 2 shows an exemplary embodiment of a device for measuring a foot shape or a foot condition according to the present invention.

FIG. 3 shows an exemplary embodiment of a process for obtaining a diagnosing data according to the present invention.

FIG. 4 shows an exemplary embodiment of an operating process of the system according to the present invention.

FIG. 5 shows an exemplary embodiment of a method for diagnosing a foot by scanning the foot according to the present invention.

### DETAILED DESCRIPTION OF EXEMPLARY EMBODIMENTS

Exemplary embodiments of the present invention will be described herein below with reference to the accompanying drawings.

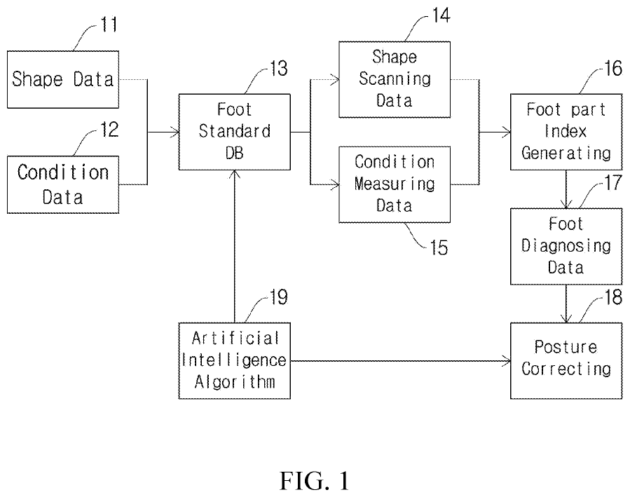

FIG. 1 shows an exemplary embodiment of a system for diagnosing a foot condition by scanning the foot according to present invention.

Referring to FIG. 1, a system for diagnosing a foot condition by scanning the foot comprises a shape data module 11 for detecting various shapes of foots and storing shape data; a condition data module 12 for detecting the foot condition or a foot related condition and storing the condition data; a foot standard database 13 for generating a standard data of the food based on the data stored in the shape data module 11 and a condition data module 12 and storing the generated standard data; a shape scanning data module 14 for generating a 3D shape data related to the foot shape by scanning the foot in 3D way; a condition measuring data module 15 for measuring a foot condition by scanning the foot and generating a foot condition data; and a foot diagnosing data module 17 for generating a foot data based on the data generated by the shape scanning data module 14 and the condition measuring data module 15.

Various foot shapes may be detected and stored by the shape data module 11, The shape data module 11 may investigate the data related to the foot shape, or collect and store the data related to the shape by scanning the foot in 3D way. The condition data module 12 may store the data according to a foot condition, and collect various data related to the foot. For example, the condition data module 21 may collect a foot pressure center according to contacting the ground, contacting pressures of various foot parts, a foot temperature, an electric conductivity of various foot parts and the like. And also, the condition data module 12 may store various disease data related to the foot. The foot standard database 13 may be made based on the data stored on the shape data module 11 and the condition data module 12. The foot standard database 13 may classify and store a standard foot shape and a standard foot condition, and store a foot data corresponding to an abnormal shape. And also, the foot standard database 13 may store a condition data according to a normal foot shape or an abnormal foot shape. And then a diagnosing dada may be generated based on the standard database. Each foot may be scanned by the shape scanning data module 14 for generating a diagnosing data related to each foot, and the shape, and the shape scanning data module 14 may comprise a scanner or a camera for obtaining a 3D scanning data of the foot. The 3D shape scanning data for generating a 3D model of the foot may be generated by the shape scanning data module 14. A condition related to various parts of a foot may be measured by the condition measuring data module 15. The condition measuring data module 15 may measure a contacting pressure of various foot parts, a stimulus response, a temperature or the like. When the 3D scanning data and the condition measuring data related to the foot is obtained, the diagnosing index to each parts of the foot may be generated. In particular, the foot may be divided into a plurality of parts, and each part may be compared with a standard data or an abnormal data stored in the standard database to generate an estimating index. Fox example, the estimating index may be expressed as a range of −10 to +10 in base of 0. And as the absolute value become larger, the abnormal condition may become more serious. The foot diagnosing data may be generated based on the estimating index to each divided parts by the foot diagnosing data module 17. And a correctable posture may be investigated based on the foot diagnosing data by the posture correcting module 18. For example, the correctable posture may be found using an artificial algorithm module 19. In particular, a standard correctable type measured from the foot standard database 13 may be investigated by the artificial algorithm module 19. And the correctable posture can be found based on the investigated posture. The artificial algorithm module 19 may investigate a proper standard data from the foot standard database 13 in various ways for delivering the proper standard data to the posture correcting module 18. And also, the posture correcting module 18 may investigate the correctable posture based on the standard data in various ways, not limited to.

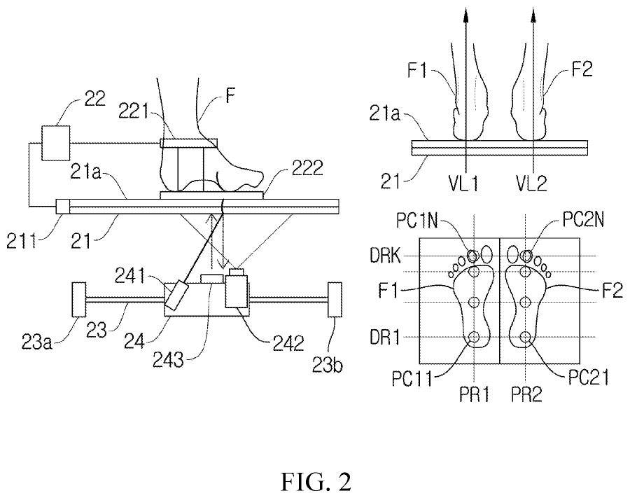

FIG. 2 shows an exemplary embodiment of a device for measuring a foot shape or a foot condition according to the present invention.

Referring to FIG. 2, the foot shape data and the foot condition data can be obtained by a foot scanning device. A data for diagnosing may be obtained by the foot scanning device, selectively a data required on manufacturing an insole for correcting a posture may be obtained. The scanning device may comprise various means for detecting a foot shape or a response to a stimulus. The scanning device may comprise a scanning module 24 for detecting the foot shape, a temperature distribution or a pressure distribution, a stimulus applying module 221 for detecting a response to a stimulus and a contacting board 21a for detecting a contacting pressure. The contacting board 21a may be installed on a base board 21 with a rectangular plate shape, and the contacting board 21a may comprise a plurality of contacting areas divided by a plurality column dividing lines and row dividing lines. And also, a sole of the foot may be divided as a plurality of divided areas by a plurality horizontal and vertical liens, and each reference point may be set at each divided area. And each reference point may contact each contact detecting area, and a contacting area size and a contacting pressure may be detected at each contact detecting area for obtaining the foot condition data. A stimulus detecting board 222 may be formed on the contacting board 21a, and the stimulus detecting board 222 may comprise a plurality of stimulus applying points separated each other. A stimulus may be applied at each stimulus applying point separated each other by the stimulus applying module 221, and for example, the stimulus may be a micro current in a 100 to 1,000 μA. The micro current applied to each stimulus applying point by the stimulus applying module 221 and induced into the human body may be detected, and the result may be transmitted to a controlling module 22. And each pressure of each divided area applied to the contacting board 21a may be detected by the detecting unit 211 and may be delivered to the controlling module 22.

The base board may be made of a stuff transparent to a light such as a laser or an infrared ray, and selectively a pressure detecting film may be attached on the base board 21. The pressure detecting film may be a conductive film operating as a capacitive type, and a plurality of girds or pixels may be arranged on the film for detecting a pressure, and a resistance according to a pressure applied on each predetermined area may be measured. A scanning module may be placed under the base board 21. The scan module 24 may move along a linear rail guide 23, and both ends of the linear rail guide 23 may be fixed at regulating blocks 25a, 25b. The position of the scan module 24 at the linear rail guide 23 may be determined by the regulating block 2 25a, 25b, and a scan data may be obtained at different points of the linear rail guide 23. The scan module 24 may comprise a laser scanner 241; a camera 242 whose focus forms at a measuring place of the laser scanner 241; and at least one condition detecting sensor 243. The condition detecting sensor 24 may be an infrared ray sensor for detecting a temperature, and each temperature of each foot part may be detected by the infrared ray sensor. And also, a temperature difference of different foot parts according to a stimulus may be detected by the condition detecting sensor 243.

Shown at the right part of FIG. 2, a condition of both Achilles tendons in both feet F1, F2 may be detected at the same time. In particular, a tilting level of Achilles tendon to reference vertical lines VL1, VL2 may be detected as ascending from the sole to the heel based on the reference vertical lines VL1, VL2. And also, a pressure center to the longitudinal direction of foot may be detected. In particular, each pressure reference line PR1, PR2 to the longitudinal direction of each foot F1, F2 may be set, and a plurality of dividing lines DR1 to RRK may be set in the vertical direction to the pressure reference lines RP1, PR2. And pressure center points PC11 to PC2N where the maximum pressure acts based on the plurality of dividing liens DR1 to DRK may be investigated, And a pressure center curve connecting the pressure center points PC11 to PC2N may be generated. Hereby the condition of the foot may be detected. Each foot F1, F2 may be divided as a plurality of areas, and the divided areas may be set based on the contacting board 21a.

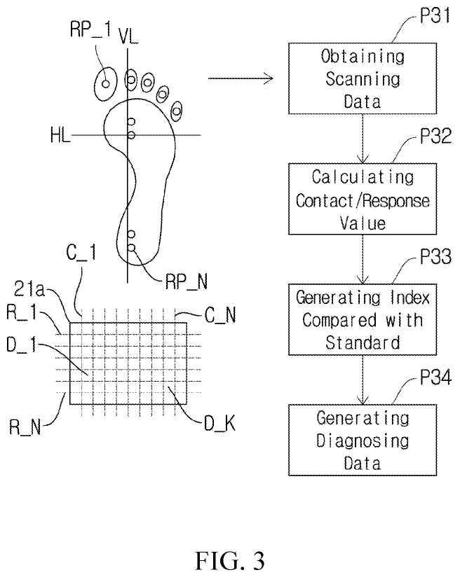

FIG. 3 shows an exemplary embodiment of a process for obtaining a diagnosing data according to the present invention.

Referring to FIG. 3, the contacting board 21a may comprise a plurality of contact detecting areas D_1 to D_K divided by a plurality of row dividing lines R_1 to R_N and a plurality of column dividing lines C_1 to C_N. And also, the sole of foot may be divided as a plurality of dividing areas by a plurality of horizontal lines and a plurality of vertical lines. Each reference points RP_1 to RP_N may be set at each dividing area of each sole. And each reference point RP_1 to RP_N may contact each contact detecting area D_1 to D_K, and each contacting area and each contacting pressure may be detected at each contacting detecting area D_1 to D_N, and thereby the condition data of the foot may be obtained. In such a state, the divided area may be set to coincide with the contact detecting area D_1 to D_K. In this set state, a scanning data may be obtained P31. The scanning data may be obtained based on each divided area, and a contacting pressure and a stimulus response value may be detected to each divided area P32. A contacting pressure and a stimulus response value of a standard foot may be prepared previously, and an estimating index may be generated by being compared with the standard contacting pressure and the standard stimulus response value P33. The estimating index to the standard value may comprise the tilting level of the Achilles tendon or the center pressure curve as mentioned above. And the diagnosing data may be generated based on such estimating index to the standard P34. Various type of estimating indices may be made based on various detecting values, not limited to.

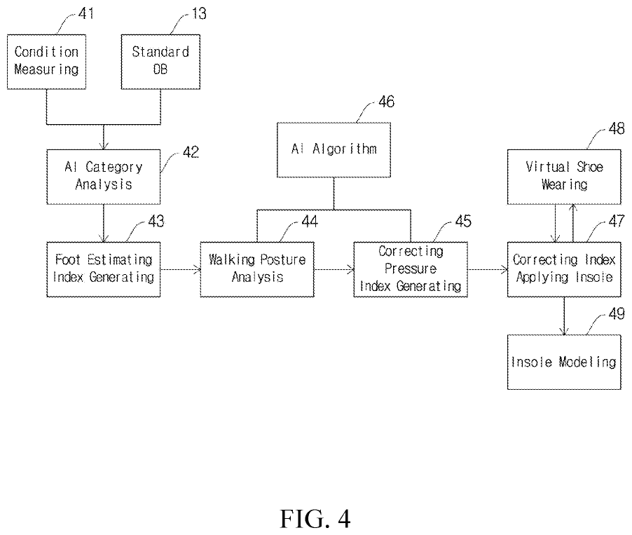

FIG. 4 shows an exemplary embodiment of an operating process of the system according to the present invention.

Referring FIG. 4, the system further comprises a foot estimating index generating module 43 for generating a foot condition index based on a reaction to a response of a pressure according to a ground contact of the foot and a stimulus. The condition of the foot may be measured by the condition measuring module 41, and the condition measuring module 41 may comprise the foot scanning device mentioned above. When the foot condition data measured by the condition measuring module 41 is obtained, the foot data corresponding to the measured foot shape may be selected from the standard database for detecting the standard condition. Such corresponding standard data may be investigated by the artificial intelligent category analyzing module 42, for example. When the standard data may be obtained by the artificial intelligent category analyzing module 42, the estimating index of the measured foot to the standard data may be generated by a foot estimating index generating module 43. The walking posture of the measured foot may be analyzed by a walking posture analyzing module 44. When the walking posture is analyzed, a correcting pressure index may be generated by a correcting pressure index generating module 45. The correcting pressure index may designate a pressure value for correcting, and the pressure value may be calculated by comparing the pressure of the measured foot with that of the standard foot. As mentioned above, the sole of the foot may comprise a plurality of divided areas, and the correcting pressure index may be calculated to each divided area. When the correcting pressure index may be generated, a correcting virtual insole that the correcting pressure is applied to may be made by a correcting index applying insole module 47. When the correcting virtual insole is made, the posture may be verified by a virtual shoe wearing module 48. And then, an insole model may be generated by an insole modeling module, if it is verified that the posture correction is proper. The insole model using the correcting pressure may be generated in various ways, not limited to.

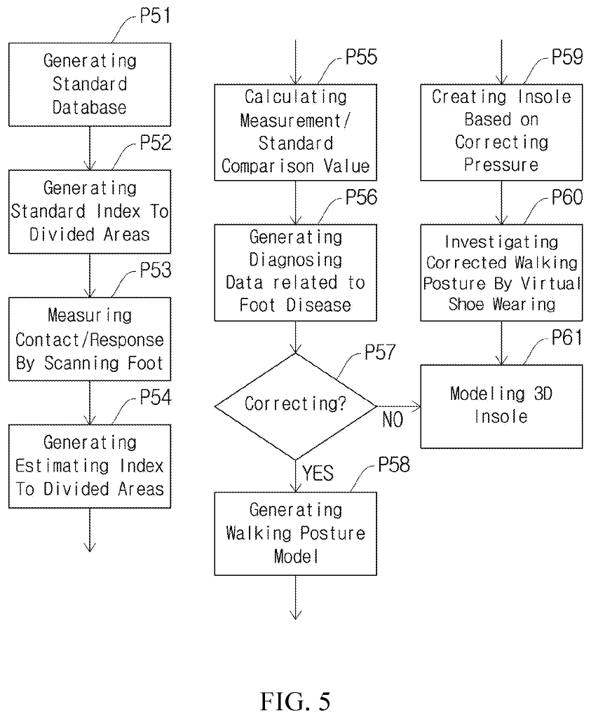

FIG. 5 shows an exemplary embodiment of a method for diagnosing a foot by scanning the foot according to the present invention.

Referring to FIG. 5, a method for diagnosing a foot by scanning the foot may comprise generating a standard database related to the foot shape or condition P51; obtaining a shape data by scanning the foot P52; measuring a contacting pressure and a stimulus response P53; generating an estimating index related to divided parts of the foot based on the contacting pressure and the stimulus response P54; comparing the estimating index with a data from the standard database P55; and generating a diagnosing data related to a foot related disease P56.

The standard database may be generated through the above-mentioned method, and a standard foot or a standard foot data corresponding to the measured foot may be selected. And the standard foot may be divided as a plurality of divided areas, and a divided part standard index to each divided area of the standard foot P52. As mentioned above, the contacting pressure and the stimulus response may be measured by the foot scanning device P54. And then, the estimating index related to the divided parts of the measured foot P54. When the measurement of the foot is completed, the estimating index of the measured foot may be compared with the standard index for calculating the difference value. And then, the diagnosing data related to the foot disease may be generated based on the difference value P56. It is determined whether the foot correction is required or not based on the diagnosing data. If the correction is not required NO, then a 3D insole model may be made. On the other hand, if the correction is required YES, then a walking posture model may be generated P58. A correcting pressure may be calculated based on the walking posture model, and a corrected insole model based on the correcting pressure may be created P59. When the virtual insole model is made P59, the virtual insole model is worn virtually for investigating the corrected walking posture P60. In this course, an artificial algorithm may be applied. And then, the 3D insole may be made based on the virtual shoe wearing P61. In a course of investigating the compatibility of the insole by the virtual shoe wearing, the artificial algorithm may be applied, not limited to.

While the present invention has been particularly shown and described with reference to exemplary embodiments thereof, it will be understood by those skilled in the art that various changes in form and details may be made therein without departing from the spirit and scope of the present invention as defined by the appended claims.

What is claimed is:

1. A system for diagnosing a foot condition by scanning the foot, comprising:
a shape data module 11 for detecting various shapes of foots and storing shape data;
a condition data module 12 for detecting the foot condition or a foot related condition and storing the condition data;
a foot standard database 13 for generating a standard data of the food based on the data stored in the shape data module 11 and a condition data module 12 and storing the generated standard data;
a shape scanning data module 14 for generating a 3D shape data related to the foot shape by scanning the foot in 3D way;
a condition measuring data module 15 for measuring a foot condition by scanning the foot and generating a foot condition data; and
a foot diagnosing data module 17 for generating a foot data based on the data generated by the shape scanning data module 14 and the condition measuring data module 15.

2. The system according to claim 1, wherein the system further comprises a foot estimating index generating module 43 for generating a foot condition index based on a reaction to a response of a pressure according to a ground contact of the foot and a stimulus.

3. A method for diagnosing a foot by scanning the foot, comprising:
generating a standard database related to the foot shape or condition;
obtaining a shape data by scanning the foot;
measuring a contacting pressure and a stimulus response;
generating an estimating index related to divided parts of the foot based on the contacting pressure and the stimulus response;
comparing the estimating index with a data from the standard database; and
generating a diagnosing data related to a foot related disease.
